(19) United States
(12) Patent Application Publication
(10) Pub. No.: US 2025/0197331 A1
(43) Pub. Date: Jun. 19, 2025

(54) AN INTEGRATED PROCESS TO PRODUCE PHENOL FROM COAL DERIVED LIQUID

(71) Applicants: UOP LLC, Des Plaines, IL (US); CHINA PETROLEUM & CHEMICAL CORPORATION, Beijing (CN)

(72) Inventors: Zheyan QIU, Sugar Land, TX (US); Somphonh Peter PHIVILAY, Brookshire, TX (US); Zhenhua ZHOU, Katy, TX (US); Neelesh RANE, Des Plaines, IL (US)

(21) Appl. No.: 18/847,757

(22) PCT Filed: Mar. 17, 2022
(86) PCT No.: PCT/US2022/071190

§ 371 Date: Sep. 17, 2024

(51) Int. Cl. C07C 37/50; C07C 6/12; C07C 37/48; C07C 37/68

(52) U.S. Cl. CPC C07C 37/50; C07C 6/12; C07C 37/48; C07C 37/68

(57) ABSTRACT

Process for producing phenol, cresol and xylenes from a coal-derived feed are described. The processes combine dealkylation of alkylphenols from coal derived liquids followed by benzene and/or toluene transalkylation to reduce the production of non-ideal alkylbenzenes and reduce the usage of benzene/toluene. Alkylphenols from the coal derived liquids are converted in a dealkylation reaction zone comprising a dealkylation reactor to make phenol. The unconverted alkylphenols and an aromatic compound, such as benzene or toluene, are fed to a transalkylation reaction zone comprising a transalkylation reactor to make more phenol. Cresols and xylenes can also be produced.

Coal derived liquids from coal gasification and coke plants contain an abundance of valuable phenolic compounds, including phenol, cresols, and xylenols, as well as less desirable long-chain alkylphenols. See e.g., Junmin Zhang, Gong Liu, “Comprehensive Utilization of Low-temperature Coal Tar”, Coal Conversion, 92-96, 33 (2010); Xiaojing Zhang, “Hydrogenation Process for Coal Tar from Mid-low-temperature Coal Carbonization”, J. of China Coal Society, 840-844, 36 (2011); Shiyu Wang, Xiaoyan Bai, Yang Zhang, Libin Wang, Sijian Qu, “The Separation of Group Composition by Column Chromatography and GC/MS Analysis” Clean Coal Technology, 59-62, 16 (2010); Xianglan Li, Xintao Cui, Yongfa Zhang, “Analysis of Low Temperature Coal Tar in Inner Mongolia Lignite Briquette by GC-MS”, Analytical Instrument, 17-25,3 (2012); Xuezhi Liu, Pang Jin, “Study on the Tar Structure and Nature if Pressurized Pyrolysis for Tianzhu Coal in Gansu”, Coal Conversion, 82-88, 17 (1994); and https://www.dakotagas.com/products/chemicals-and-fuels/dephenolized-cresylic-acid. Long-chain alkylphenols have alkyl groups with 2 or more carbon atoms (e.g., ethylphenols, propylphenols, butylphenols, methylethyl phenols, and the like).

The long-chain alkylphenols can be converted into phenol and ethylene and/or propylene through dealkylation. The catalytic vapor phase hydrodealkylation of low temperature tar acid fractions (170° C.˜270° C.) to low boiling phenols was studied in a flow system in the temperature range 480-650° C., and over a wide range of space velocities on a chromia-alumina catalyst at atmospheric pressure. See K. K. Tiwari, S. N. Banerji, Asit Bhattacharjee and R. N. Bhattacharya, “Catalytic Hydrodealkylation of Tar Acids”, Applied Catalysis, 39-51, 45 (1988).

As discussed in Danny Verboekend, Yuhe Liao, Wouter Schutyser and Bert F. Sels, “Alkylphenols to phenol and olefins by zeolite catalysis: A pathway to valorize raw and fossilized lignocellulose”, Green Chem., 2016 (1), 18, 297-306; and Yuhe Liao, Ruyi Zhong, Ekaterina Makshina, etc., “Propylphenol to Phenol and Propylene over Acidic Zeolites: Role of Shape Selectivity and Presence of Steam”, ACS Catal. 2018, 8, 9, 7861-7878, the phenol yield of liquid product ranges from 26.8 wt % to 55.2 wt % for the different cuts at 600° C. However, the phenol itself in the feed contributes the yields, leading to incorrect results. Hence, pre-separation may be needed. Ethylphenol and propylphenol, can be highly-selectively converted to relevant base chemicals phenol and corresponding olefin. Highly efficient and robust dealkylation (95% selectivity of phenol and propylene) is achieved using ZSM-5 zeolite catalysts and the co-feeding of water/steam.

A full isopropylphenol conversion with over 99% phenol selectivity using the Y-zeolite at 350° C. was obtained, as described in Shogo Kumagai, Masaki Asakawa, Tomohito Kameda, Yuko Saito, Atsushi Watanabe, Chuichi Watanabe, Norio Teramae & Toshiaki Yoshioka, “Selective phenol recovery via simultaneous hydrogenation/dealkylation of isopropyl- and isopropenyl-phenols employing an H₂ generator combined with tandem micro-reactor GC/MS”, Scientific Reports, 11-12,8 (2018).

The long-chain alkylphenols may be transalkylated with an aromatic solvent such as benzene/toluene to transfer the long-chain alkyl group (ethyl, propyl, butyl, etc.) to obtain phenol and alkylbenzenes. Transalkylation of 4-propylphenol with benzene was explored using HZSM-5 at 350° C. It was found that the major products from benzene translakylation were n-propylbenzene (58 mol %) and cumene (28 mol %). Xiaoming Huang, Jasper M. Ludenhoff, Mike Dirks, Xianhong Ouyang, Michael D. Boot, and Emiel J. M. Hensen, “Selective Production of Biobased Phenol from Lignocellulose Derived Alkylmethoxyphenols”, ACS Catalysis, 11184-11190, 8 (2018).

Another study for transalkylation of p-propylphenol with benzene over zeolite catalysts at 400° C. and 11.6 MPa shows that phenol yield can reach 22 mole % after 8 hours running. The main products of transalkylation with benzene were alkylbenzenes. Takuya Yoshikawa, Takahiro Umezawa, Yuta Nakasaka and Takao Masuda, “Conversion of alkylphenol to phenol via transalkylation using zeolite catalysts”, Catalysis Today, 110-114, 347 (2020). However, this is not suitable when the market has a higher demand for phenol than for alkylbenzenes.

US2020/0031741 describes a process to make xylenes and phenol by transalkylation of coal derived liquids with benzene and/or toluene. Phenol, xylenols and cresols and long-chain alkylphenols are pre-separated from the coal tar in this process. However, feeding cresol into a transalkylation unit to make phenol and xylene is not desirable because cresol is a higher value product than phenol. No details about how to process long-chain alkylphenols are provided.

WO2020162877 describes another process to produce phenol and xylenes from a phenol containing feed. In this invention, all alkylphenols except phenol are transalkylated by benzene and/or toluene. There is a same issue as in US2020/0031741 for the high value cresol component. Additionally, a large quantity of benzene and/or toluene is needed for transalkylation of long-chain alkylphenols with large amount of alkylbenzene product made. The size of and the downstream processing equipment is relatively large and not cost effective.

WO2020/162876 describes an integrated process of dealkylation and transalkylation to make cresols from a phenol containing feed, but the desired products from this process are cresols. Although phenol and cresols are pre-separated, xylenols are sent with other heavy alkylphenols for dealkylation, which results in the potential cracking of xylenols. Moreover, reactors for the process are large. In addition, some aromatics can be produced during dealkylation, and their separation from alkylphenols is not addressed.

Therefore, there remains a need for processes for converting long-chain phenols from coal derived feeds into more valuable products such as phenols.

### BRIEF DESCRIPTION OF THE DRAWING

FIG. 1 is a schematic of one embodiment of a process according to the present invention.

FIG. 2 is a schematic of another embodiment of a process according to the present invention.

### DESCRIPTION

Novel processes for producing phenol, cresol and xylenes from a coal-derived feed have been developed integrating dealkylation of alkylphenols from coal derived liquids with transalkylation using benzene and/or toluene. The alkylphenols from the coal derived liquids are converted to phenol by dealkylation. The unconverted alkylphenols and aromatic compounds, such as benzene or toluene, are transalkylated to make more phenol. Cresols and xylenes can also be produced. These processes maximize phenol product yield and decrease alkylbenzene yield.

Initial work involving toluene transalkylation with model components 4-ethylphenol and 2,6-xylenol showed that alkylbenzenes (xylenes, ethylbenzene, ethyltoluenes, etc.) have higher selectivity than phenol when the molar ratio of toluene/alkylphenols is 6:1. Process simulations show that the toluene transalkylation of alkylphenolic compounds of the acid fraction from Xijiang Hami low/medium coal tar (coal from Xingjiang Hami, CN) can produce about 56 wt % of alkylbenzenes and 11.5 wt % of phenol.

One concern with a stand-alone transalkylation process is that multiple low value alkylbenzene compounds, such as ethyltoluene, and n-propylbenzene, are produced from long-chain alkylphenols. These alkylbenzene products have low market demand. A process starting with a dealkylation reaction makes phenol as the main product from ethylphenols, propylphenols, and ethylmethylphenols without producing the low value alkylbenzenes. However, xylenols and trimethylphenols have very poor reactivity for dealkylation reaction. By integrating the transalkylation into the process as second reaction, xylenols and trimethylphenols can react with toluene to make phenol, cresols and xylenes. In this way, it is not only avoiding the production of less desired alkylbenzenes, the reactor size and toluene consumption for the transalkylation reaction are reduced significantly.

Additional testing has shown that dealkylation of ethylphenol using an HZSM-5 catalyst has higher phenol selectivity.

By combining benzene or toluene transalkylation with dealkylation, it is possible to maximize phenol production and minimize alkylbenzene product. The long-chain alkylphenols (e.g., ethylphenols, propylphenols, butylphenols, etc.) are dealkylated into phenol and olefins. The short-chain alkylphenols (having alkyl groups with one carbon atom, such as xylenol, trimethylphenol, and the like) are transalkylated into phenol and cresol with alkylbenzenes as a byproduct.

The present processes combine dealkylation of long-chain alkylphenols from coal derived liquids followed by benzene and/or toluene transalkylation to reduce the production of less desired alkylbenzenes. Long-chain alkylphenols from the coal derived liquids are converted in the dealkylation reaction zone comprising a dealkylation reactor to make phenol. The unconverted alkylphenols together with xylenols and an aromatic compound, such as benzene or toluene or a mixture of them, are fed to a transalkylation reaction zone comprising a transalkylation reactor to make more phenol. In some embodiments, toluene is chosen as the solvent because benzene has higher toxicity. Unconverted toluene, benzene, and other aromatics can be recycled back to the transalkylation reaction zone to reduce the toluene and/or benzene makeup rate.

The present processes involve separation of phenol and cresol from the alkylphenols from coal derived liquids prior to dealkylation and transalkylation. This can prevent phenol and cresol from cracking into less valuable products in the dealkylation step and can also reduce the size of the downstream processes and equipment.

In one embodiment, the present process involves separation of xylenols from the alkylphenols from coal derived liquids prior to dealkylation and transalkylation. This can prevent xylenols from cracking into less valuable products in the dealkylation step and can also reduce the size of the downstream processes and equipment.

One aspect of the invention is a process for producing phenol from a coal-derived feed. In one embodiment, the process comprises: providing an alkylphenol stream comprising alkylphenols having an alkyl chain having two or more carbon atoms; dealkylating the alkylphenol stream in a first dealkylation reaction zone comprising a first dealkylation reactor under first dealkylation reaction conditions in the presence of a first dealkylation catalyst to produce a first dealkylation effluent stream comprising light hydrocarbons, alkylbenzene, phenol, cresol, and unconverted alkylphenol; separating the first dealkylation effluent stream in a dealkylation separation zone into at least a phenol stream comprising the phenol and a bottom stream comprising the unconverted alkylphenol; transalkylating the bottom stream and a reactant stream comprising toluene or benzene or both in a first transalkylation reaction zone comprising a first transalkylation reactor under first transalkylation reaction conditions in the presence of a first transalkylation catalyst to produce a first transalkylation effluent stream comprising cresol, phenol, and alkylbenzene; introducing the first transalkylation effluent stream into the dealkylation separation zone; and recovering the phenol stream.

In some embodiments, separating the first dealkylation effluent stream in the dealkylation separation zone comprises separating the first dealkylation effluent stream into the phenol stream, the bottom stream, and at least one of a cresol stream comprising the cresol, a light hydrocarbon stream comprising the light hydrocarbons, and an alkylbenzene stream comprising the alkylbenzene.

In some embodiments, the process further comprises: separating the cresol stream in a cresol separation zone into a mixed cresol stream comprising p-cresol and o-cresol, and a m-cresol stream comprising m-cresol; isomerizing the mixed cresol stream in a cresol isomerization reaction zone under cresol isomerization reaction conditions in the presence of a cresol isomerization catalyst to form a cresol isomerization effluent stream comprising a mixture of cresol; recycling the cresol isomerization effluent stream to the cresol separation zone; and recovering the m-cresol stream comprising m-cresol.

In some embodiments, the process further comprises: separating the alkylbenzene stream in an alkylbenzene separation zone into at least a recycle stream comprising one or more of benzene and toluene, a heavy alkylbenzene stream comprising alkylbenzene having an alkyl chain having two or more carbon atoms, and a mixed xylene stream comprising a mixture of xylenes and ethylbenzene; and recycling the recycle stream from the alkylbenzene separation zone to the first transalkylation reaction zone.

In some embodiments, the process further comprises: separating the mixed xylene stream in a xylene separation zone into a second xylene stream comprising ethylbenzene, o-xylene, and m-xylene, and a p-xylene stream comprising p-xylene; isomerizing the second xylene stream in a xylene isomerization reaction zone under xylene isomerization reaction conditions in the presence of a xylene isomerization catalyst to form a xylene isomerization effluent stream comprising a mixture of xylene; recycling the xylene isomerization effluent stream to the xylene separation zone; and recovering the p-xylene stream.

In some embodiments, the process further comprises: dealkylating the heavy alkylbenzene stream in a second dealkylation reaction zone comprising a second dealkylation reactor under second dealkylation conditions in the presence of a second dealkylation catalyst to form a dealkylated heavy alkylbenzene effluent stream comprising xylene, and benzene or toluene or both; or transalkylating the heavy alkylbenzene stream in a second transalkylation reaction zone comprising a second transalkylation reactor under second transalkylation conditions in the presence of a second transalkylation catalyst to form a transalkylated heavy alkylbenzene effluent stream comprising xylene and benzene or toluene or both; and optionally recycling the dealkylated heavy alkylbenzene effluent stream, the transalkylated heavy alkylbenzene effluent stream, or both to the alkylbenzene separation zone.

In some embodiments, the bottom stream from the dealkylation separation zone further comprises toluene or xylenol or both.

In some embodiments, providing the alkylphenol stream comprises: providing a coal-derived feed stream to a first separation zone; and separating the coal-derived feed stream in the first separation zone into the alkylphenol stream and at least one of a first phenol stream comprising phenol, and a first cresol stream comprising cresol.

In some embodiments, separating the coal-derived feed stream comprises separating the coal-derived feed stream by one or more of extraction, absorption, extractive distillation, crystallization, or membrane separation.

In some embodiments, the coal-derived feed stream comprises a portion of a low temperature coal tar stream, a medium temperature coal tar stream, a high temperature coal tar stream, a cresylic acid stream, or a crude phenolic mixture.

In some embodiments, providing the alkylphenol stream comprises: providing a coal-derived feed stream to a first separation zone; and separating the coal-derived feed stream into phenol, cresol and the other alkylphenol streams.

In some embodiments, the first dealkylation reaction conditions comprise one or more of: a temperature in a range of 50° C. to 700° C.; a pressure in a range of 0 MPa (g) to 5 MPa (g); a WHSV in a range of 0.1 to 300 hr⁻¹.

In some embodiments, the first transalkylation reaction conditions comprise one or more of: a temperature in a range of 50° C. to 600° C.; a pressure in a range of 0 MPa (g) to 15 MPa (g); and a WHSV in a range of 0.1 to 300 hr⁻¹.

In some embodiments, dealkylating the alkylphenol stream and the reactant stream takes place in the presence of steam, nitrogen, or a combination thereof in the dealkylation reaction zone.

In some embodiments, transalkylating the bottom stream and the reactant stream takes place in the presence of hydrogen, nitrogen, steam, or a combination thereof in the first transalkylation reaction zone.

In some embodiments, the dealkylation catalyst comprises a heterogeneous acid catalyst, or wherein the first transalkylation catalyst comprises a heterogeneous acid catalyst, or both.

In some embodiments, at least one of the first dealkylation reaction zone and the first transalkylation reaction zone comprises a fixed bed reactor, a moving bed reactor, an ebullated bed reactor, a fluidized bed reactor, a continuous catalyst regeneration (CCR) reactor, a semi-regenerative reactor, a batch reactor, a continuous stirred tank (CSTR) reactor, a slurry reactor, or combinations thereof.

Another aspect of the invention is a process for producing phenol from a coal-derived feed. In one embodiment, the process comprises: providing an alkylphenol stream comprising alkylphenols having an alkyl chain having two or more carbon atoms; dealkylating the alkylphenol stream in a first dealkylation reaction zone comprising a first dealkylation reactor under first dealkylation reaction conditions in the presence of a first dealkylation catalyst to produce a first dealkylation effluent stream comprising light hydrocarbons, alkylbenzene, phenol, cresol, and unconverted alkylphenol; separating the first dealkylation effluent stream in a dealkylation separation zone into at least a phenol stream comprising the phenol, a bottom stream comprising the unconverted alkylphenol, and at least one of a cresol stream comprising the cresol, a light hydrocarbon stream comprising the light hydrocarbons, and an alkylbenzene stream comprising the alkylbenzene; transalkylating the bottom stream and a reactant stream comprising toluene or benzene or both in a first transalkylation reaction zone comprising a first transalkylation reactor under first transalkylation reaction conditions in the presence of a first transalkylation catalyst to produce a first transalkylation effluent stream comprising cresol, phenol, and alkylbenzene; introducing the first transalkylation effluent stream into the dealkylation separation zone; and recovering the phenol stream.

In some embodiments, the process further comprises: separating the cresol stream from the dealkylation separation zone in a cresol separation zone into a mixed cresol stream comprising p-cresol and o-cresol, and a m-cresol stream comprising m-cresol; isomerizing the mixed cresol stream in a cresol isomerization reaction zone under cresol isomerization reaction conditions in the presence of a cresol isomerization catalyst to form a cresol isomerization effluent stream comprising a mixture of cresol; recycling the cresol isomerization effluent stream to the cresol separation zone; recovering the m-cresol stream comprising m-cresol; separating the alkylbenzene stream from the dealkylation separation zone in an alkylbenzene separation zone into at least a recycle stream comprising one or more of benzene and toluene, a heavy alkylbenzene stream comprising alkylbenzene having an alkyl chain having 2 or more carbon atoms, and a mixed xylene stream comprising a mixture of xylenes and ethylbenzene; recycling the recycle stream from the alkylbenzene separation zone to the first transalkylation reaction zone; separating the mixed xylene stream in a xylene separation zone into a second xylene stream comprising ethylbenzene, o-xylene, and m-xylene, and a p-xylene stream comprising p-xylene; isomerizing the second xylene stream in a xylene isomerization reaction zone under xylene isomerization reaction conditions in the presence of a xylene isomerization catalyst to form a xylene isomerization effluent stream comprising a mixture of xylene; recycling the xylene isomerization effluent stream to the xylene separation zone; and recovering the p-xylene stream; dealkylating the heavy alkylbenzene stream in a second dealkylation reaction zone comprising a second dealkylation reactor under second dealkylation conditions in the presence of a second dealkylation catalyst to form a dealkylated heavy alkylbenzene effluent stream comprising xylene, and benzene or toluene or both; or transalkylating the heavy alkylbenzene stream in a second transalkylation reaction zone under second transalkylation conditions in the presence of a second transalkylation catalyst to form a transalkylated heavy alkylbenzene effluent stream comprising xylene and benzene or toluene or both; and recycling the dealkylated heavy alkylbenzene effluent stream, the transalkylated heavy alkylbenzene effluent stream, or both to the alkylbenzene separation zone.

In some embodiments, providing the alkylphenol stream comprises: providing a coal-derived feed stream to a first separation zone; and separating the coal-derived feed stream in the first separation zone into the alkylphenol stream and at least one of a first phenol stream comprising phenol, and a first cresol stream comprising cresol.

In some embodiments, the first dealkylation reaction conditions comprise one or more of: a temperature in a range of 50° C. to 700° C.; a pressure in a range of 0 MPa (g) to 5 MPa (g); a WHSV in a range of 0.1 to 300 hr⁻¹; or wherein the first transalkylation reaction conditions comprise one or more of: a temperature in a range of 50° C. to 600° C.; a pressure in a range of 0 MPa (g) to 15 MPa (g); and a WHSV in a range of 0.1 to 300 hr⁻¹; or both.

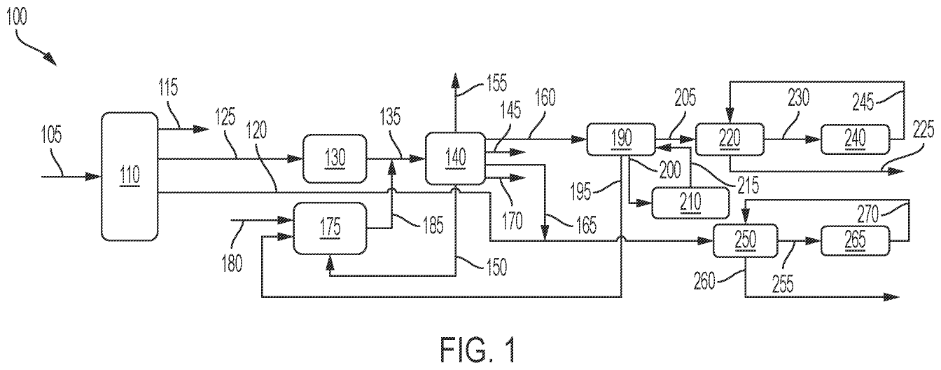

The schematic flow diagram of one embodiment of the integrated process 100 is shown in FIG. 1. A feed stream 105 comprising the acid fraction and crude phenols from coal derived liquids is sent to a first separation zone 110 where it is separated into a phenols stream 115 comprising phenol, a cresol stream 120 comprising cresol, and an alkylphenol stream 125 comprising alkylphenols having alkyl chains having two or more carbon atoms. Suitable first separation processes include, but are not limited to, distillation, extraction, adsorption, extractive distillation, crystallization, membrane separation, or combinations thereof.

The alkylphenol stream 125 is sent to a first dealkylation reaction zone 130 comprising one or more dealkylation reactors with a dealkylation catalyst.

Dealkylation can be done with or without catalyst. Alkylphenols can be dealkylated through thermal cracking at high temperature without catalyst. Dealkylation without a catalyst can be quite energy intensive because the temperature is in the range of 400 to 900° C., often 700 to 900° C. Furthermore, it is often not selective due to the loss of the hydroxyl group.

Catalytic dealkylation of heavy alkylphenols can be done at much milder conditions. Typical temperatures range from 100 to 700° C., or 200 to 540° C. Ethylphenols and propylphenols can be dealkylated at temperatures from 300 to 400° C., for example, to produce phenol and ethylene/propylene on a ZSM-5 zeolite. Debutylation of alkylphenols has also been reported on acidic clay catalyst. Any suitable dealkylation catalyst can be used, including, but not limited to, silica alumina, acidic clay, zeolites, gamma alumina, chromium oxide, other metal oxides or mixed metal oxides, or combinations thereof.

Pressures for dealkylation are generally in the range of 1-5 MPa (a). Dealkylation reactions can also performed under vacuum, for example, typically 50 kPa (a), with a maximum of 20 kPa (a). The weight hourly space velocity (WHSV) typically ranges from 1 to 5 hr⁻¹.

Water/steam may be co-fed to prevent severe catalyst deactivation. Dealkylation is normally conducted in superheated steam. Typical steam to alkylphenol weight ratios range from 0.1:1 to 10:1, or 0.5:1 to 5:1.

The first dealkylation effluent stream 135 comprises light hydrocarbons (C1-C7 hydrocarbons, e.g., ethylene, propylene, etc.), alkylbenzene, phenol, cresol, and unconverted alkylphenol.

The first dealkylation effluent stream 135 is separated in a dealkylation separation zone 140 into at least a phenol stream 145 comprising phenol and a bottom stream 150 comprising unconverted alkylphenol. The first dealkylation effluent stream 135 can optionally be separated into one or more additional streams, such as a light hydrocarbon stream 155 comprising light hydrocarbons, an alkylbenzene stream 160 comprising alkylbenzenes, a cresol stream 165 comprising cresols, and a process water stream 170 comprising water. Suitable dealkylation separation processes include, but are not limited to, distillation, extractive distillation, adsorption, crystallization, membrane separation, or combinations thereof.

The phenol stream 145 from the first dealkylation separation zone 140 can be recovered.

The bottom stream 150 is sent to a first transalkylation reaction zone 175 where it is transalkylated with a reactant feed stream 180 comprising benzene and/or toluene. The first transalkylation reaction zone 175 comprises one or more transalkylation reactors.

When a catalyst is used for transalkylation of alkylphenols, the temperature is typically in the range of 50-600° C., or 100-500° C. The transalkylation zone is typically operated at pressures ranging from about 0 MPa (g) to 15 MPa (g), or 2 MPa (g) to 11 MPa (g). The weight hourly space velocity (WHSV) is generally in the range of 0.1 to 300 hr⁻¹, or 1 to 250 hr⁻¹.

The catalyst is typically selected to have relatively high stability at a high activity level. Suitable transalkylation catalysts include, but are not limited to zeolites, acidic clay, silica alumina, acidic resins, mixed metal oxides, and the like as are known in the art.

Ratios of benzene/toluene to phenol (molar ratio) is 0.1:1 to 20:1, or 0.5:1 to 10:1, or 1:1 to 5:1.

The first transalkylation effluent stream 185 which comprises cresol, phenol, and alkylbenzene is sent to the dealkylation separation zone 140 for separation.

The alkylbenzene stream 160 can be sent to an alkylbenzene separation zone 190 where it is separated into at least a recycle stream 195 comprising one or more of benzene and toluene, a heavy alkylbenzene stream 200 comprising alkylbenzene having an alkyl chain having two or more carbon atoms, and a mixed xylene stream 205 comprising a mixture of xylenes and ethylbenzene. Suitable alkylbenzene separation processes include, but are not limited to, distillation, extractive distillation, or combinations thereof.

The recycle stream 195 may be sent to the first transalkylation reaction zone 175.

The heavy alkylbenzene stream 200 may be sent to a second dealkylation reaction zone or a second transalkylation reaction zone 210 or both where the heavy alkylbenzene stream 200 is dealkylated or transalkylated.

The heavy alkylbenzene dealkylation reaction conditions include a temperature is typically in the range of 0° C. to 600° C., or 100° C. to 500° C. The heavy alkylbenzene dealkylation zone is typically operated at pressures ranging from about 0 MPa (g) to 7.6 MPa (g), or 0.01 MPa (g) to 5 MPa (g). The WHSV is generally in the range of 0.01 to 200 hr⁻¹, or 0.1 to 100 hr⁻¹. Any suitable heavy alkylbenzene dealkylation catalyst can be used. The catalyst is typically selected to have relatively high stability at a high activity level. Suitable heavy alkylbenzene dealkylation catalysts include, but are not limited to zeolites, acidic clay, silica alumina, acidic resins, mixed metal oxides, and the like, as are known in the art.

The heavy alkylbenzene transalkylation reaction conditions include a temperature is typically in the range of 0° C. to 600° C., or 100° C. to 500° C. The heavy alkylbenzene transalkylation is typically operated at pressures ranging from about 0 MPa (g) to 15 MPa (g), or 1 MPa (g) to 11 MPa (g). The WHSV is generally in the range of 0.1 to 300 hr⁻¹, or 0.2 to 200 hr⁻¹. Any suitable heavy alkylbenzene transalkylation catalyst can be used. The catalyst is typically selected to have relatively high stability at a high activity level. Suitable heavy alkylbenzene transalkylation catalysts include, but are not limited to zeolites, acidic clay, silica alumina, acidic resins, mixed metal oxides, and the like as are known in the art.

The dealkylated effluent stream 215 comprises one or more of benzene, toluene, and xylenes, and/or the transalkylated effluent stream 215 comprises one or more of benzene, toluene, and xylenes.

The mixed xylene stream 205 may be sent to a xylene separation zone 220 where it is separated into a p-xylene stream 225 comprising p-xylene and a second mixed xylene stream 230 comprising ethylbenzene, o-xylene, and m-xylene. The p-xylene stream 225 can be recovered.

Any suitable xylenes separation process can be used, including but not limited to, distillation, adsorption (for example, UOP's Parex™ process), crystallization (for example, BP/CBI's pX process), or combinations of these processes.

The second mixed xylene stream 230 is sent to a xylene isomerization zone 240 for isomerization of the o-xylene and m-xylene. The xylene isomerization effluent stream 245 comprising a mixture of xylenes is sent to the xylene separation zone 220. The xylene isomerization zone 240 comprises one or more xylene isomerization reactors.

Suitable xylenes isomerization processes include, but are not limited to, UOP's Isomar™ process.

The xylene isomerization reaction zone 240 normally operates at reaction conditions including a temperature in the range of 50° C. to 600° C., or 100° C. to 500° C. The xylenes isomerization zone is typically operated at moderately elevated pressures broadly ranging from 0 MPa to 7.6 MPa gauge, or 0.01 MPa to 5 MPa gauge. The xylenes isomerization reaction can be effected over a wide range of liquid hourly space velocities (LHSV). The LHSV is generally in the range of from 0.1 to 5 hr⁻¹, or 0.2 to 4 hr⁻¹. In some embodiments, the xylenes isomerization reaction takes place under reactions conditions comprising a temperature in a range of 50° C. to 600° C.; a pressure in a range of 0 MPa (g) to 7.6 MPa (g); and a LHSV in a range of 0.1 to 5 hr⁻¹. In some embodiments, the xylenes isomerization reaction takes place under reactions conditions comprising a temperature in a range of 100° C. to 500° C.; a pressure in a range of 0.01 MPa (g) to 5 MPa (g); and a LHSV in a range of 0.2 to 4 hr⁻¹.

Any suitable xylenes isomerization catalyst can be used. Suitable xylenes isomerization catalysts include, but are not limited to, both homogeneous catalysts, such as BF₃—HF, and heterogeneous catalysts, such as amorphous silica alumina, zeolites or metal promoted zeolites. The catalyst is typically selected to have relatively high stability at a high activity level.

The cresol stream 165 may be sent to a cresol separation zone 250 where it is separated into a mixed cresol stream 255 comprising p-cresol and o-cresol, and a m-cresol stream 260 comprising m-cresol. The cresol stream 120 may also be sent to cresol separation zone 250. The m-cresol stream 260 can be recovered.

Suitable cresol separation processes include, but are not limited to distillation, crystallization, adsorption (for example UOP's Cresex™ adsorption process), or combinations thereof.

The mixed cresol stream 255 is sent to a cresol isomerization zone 265 for isomerization of the p-cresol and o-cresol. The cresol isomerization effluent stream 270 comprising a mixture of cresols is sent to the cresol separation zone 250.

The cresol isomerization reaction conditions typically include a temperature in the range of 50° C. to 600° C., or 100° C. to 500° C. The cresol isomerization zone is typically operated at pressures ranging from about 0 MPa (g) to 15 MPa (g), or 0.01 MPa (g) to 10 MPa (g). The WHSV is generally in the range of 0.1 to 200 hr⁻¹, or 0.2 to 100 hr⁻¹.

Any suitable cresol isomerization catalyst can be used. The cresol isomerization catalyst is typically selected to have relatively high stability at a high activity level. Suitable cresol isomerization catalysts include, but are not limited to zeolites, acidic clay, silica alumina, acidic resins, mixed metal oxides, and the like, as are known in the art.

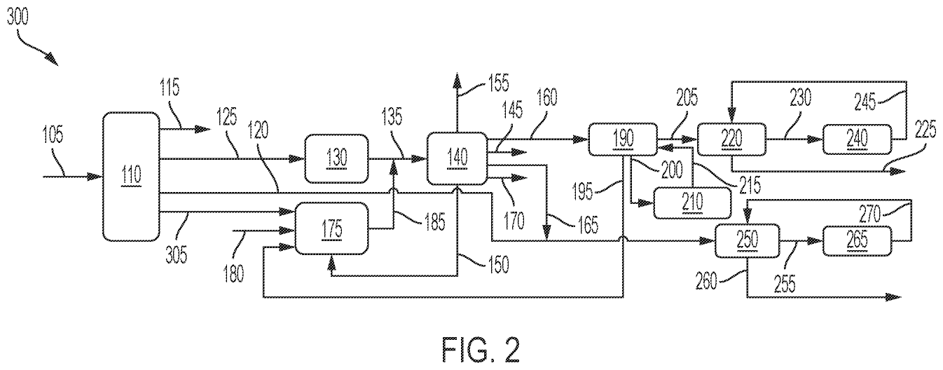

FIG. 2 illustrates another embodiment the integrated process 300. The process 300 is similar to the process 100 shown in FIG. 1. The difference is that the feed stream 105 is separated in the first separation zone 110 into the phenols stream 115, the cresol stream 120, the alkylphenol stream 125, and a xylenols stream 305 comprising xylenols and trimethylphenol. The xylenols stream 305 may be sent to the first transalkylation reaction zone 175 along with the bottom stream 150 from the dealkylation separation zone 140, the reactant feed stream 180, and the recycle stream 195 from the alkylbenzene separation zone 190. The xylenols are converted into phenol and cresol in the first transalkylation reaction zone 175.

The process of FIG. 2 (with the co-feed of the xylenols stream from the first separation zone 110 directly into the first transalkylation reaction zone 175) can reduce the equipment size of dealkylation process. It also provides more flexibility for plant operation to meet market need for xylenols product.

### EXAMPLES

The predicted product yields of toluene transalkylation and dealkylation from computer simulations using Honeywell UniSim Design software are summarized in Table 1. The calculation assumes 98% overall conversion for dealkylation and 95% toluene recycle ratio for transalkylation.

The product profile of dealkylation is based on alkylphenols only and no xylenol in the feed.

TABLE 1
Product profile of proposed dealkylation and
toluene transalkylation of alkylphenols

| Product | | | Toluene | |
|---|---|---|---|---|
| profile, | | | transalkylation | Toluene |
| wt % | Feed | Dealkylation | without xylenol | transalkylation |
| Light | | 1.22 | 0.00 | 0.00 |
| hydrocarbon | | | | |
| (C₁-C₇) | | | | |
| C₂/C₃ olefins | | 8.09 | 0.00 | 0.00 |
| Alkylbenzenes | | 1.91 | 39.54 | 56.50 |
| Phenol | | 54.62 | 21.12 | 11.50 |
| Cresol | | 0.68 | 1.00 | 6.68 |
| Xylenol | 35.39 | 0 | 0.81 | 3.49 |
| Alkylphenols | 57.24 | 17.85 | 31.6 | 19.29 |
| (with alkyl ≥ 2) | | | | |
| Others | 7.37 | 15.62 | 5.94 | 2.55 |
| (naphthols) | | | | |

The predicted product profiles of the proposed integrated process are compared with toluene transalkylation only in Table 2 using Honeywell UniSim Design. The phenol yield is increased by up to about 26 wt % by the integrated process. The phenol yield of integrated case (Case 1) is increased by about 100% compared to toluene transalkylation case (Case 0). When all xylenols from the first separation zone are claimed as products and will not join the transalkylaiton with toluene, the phenol yield is maximized. The yield of alkylbenzenes is reduced from 56.5 wt % of Case 0 to 15 wt % in Case 2. Hence, phenol becomes the main product rather than alkylbenzenes in Case 2, which is desirable for phenol plant.

TABLE 2
Product profiles of dealkylation and alkylphenolics
toluene transalkylation integration process

| | | Case 1 | Case 2 |
|---|---|---|---|
| | Case 0 | (Dealkylation | (Dealkylation |
| | (Toluene | and toluene | and toluene |
| Product | transalkylation | Transalkylation | transalkylation |
| profile, wt % | only) | with xylenol) | without xylenol) |
| Light hydrocarbon | 0.00 | 0.40 | 0.66 |
| C2/C3 olefins | 0.00 | 2.63 | 4.39 |
| Alkylbenzene | 56.50 | 44.81 | 15.15 |
| Phenol | 11.50 | 24.49 | 37.13 |
| Cresol | 6.68 | 6.07 | 0.73 |
| Xylenol | 3.49 | 3.49 | 29.40 |
| Alkylphenols(with | 19.29 | 14.46 | 6.46 |
| alkyl >= 2) | | | |
| Others(naphthols) | 2.55 | 3.65 | 6.08 |

As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones.

As used herein, the term “about” means with 10% of the value, or within 5%, or within 1%.

As depicted, process flow lines in the figures can be referred to, interchangeably, as, e.g., lines, pipes, branches, distributors, streams, effluents, feeds, products, portions, catalysts, withdrawals, recycles, suctions, discharges, and caustics.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A process for producing phenol from a coal-derived feed comprising:
providing an alkylphenol stream comprising alkylphenols having an alkyl chain having two or more carbon atoms;
dealkylating the alkylphenol stream in a first dealkylation reaction zone comprising a first dealkylation reactor under first dealkylation reaction conditions in the presence of a first dealkylation catalyst to produce a first dealkylation effluent stream comprising light hydrocarbons, alkylbenzene, phenol, cresol, and unconverted alkylphenol;
separating the first dealkylation effluent stream in a dealkylation separation zone into at least a phenol stream comprising the phenol and a bottom stream comprising the unconverted alkylphenol;
transalkylating the bottom stream and a reactant stream comprising toluene or benzene or both in a first transalkylation reaction zone comprising a first transalkylation reactor under first transalkylation reaction conditions in the presence of a first transalkylation catalyst to produce a first transalkylation effluent stream comprising cresol, phenol, and alkylbenzene;
introducing the first transalkylation effluent stream into the dealkylation separation zone; and
recovering the phenol stream.

2. The process of claim 1 wherein separating the first dealkylation effluent stream in the dealkylation separation zone comprises separating the first dealkylation effluent stream into the phenol stream, the bottom stream, and at least one of a cresol stream comprising the cresol, a light hydrocarbon stream comprising the light hydrocarbons, and an alkylbenzene stream comprising the alkylbenzene.

3. The process of claim 2 further comprising:
separating the cresol stream in a cresol separation zone into a mixed cresol stream comprising p-cresol and o-cresol, and a m-cresol stream comprising m-cresol;
isomerizing the mixed cresol stream in a cresol isomerization reaction zone under cresol isomerization reaction conditions in the presence of a cresol isomerization catalyst to form a cresol isomerization effluent stream comprising a mixture of cresol;
recycling the cresol isomerization effluent stream to the cresol separation zone; and
recovering the m-cresol stream comprising m-cresol.

4. The process of claim 2 further comprising:
separating the alkylbenzene stream in an alkylbenzene separation zone into at least a recycle stream comprising one or more of benzene and toluene, a heavy alkylbenzene stream comprising alkylbenzene having an alkyl chain having two or more carbon atoms, and a mixed xylene stream comprising a mixture of xylenes and ethylbenzene; and
recycling the recycle stream from the alkylbenzene separation zone to the first transalkylation reaction zone.

5. The process of claim 4 further comprising:
separating the mixed xylene stream in a xylene separation zone into a second xylene stream comprising ethylbenzene, o-xylene, and m-xylene, and a p-xylene stream comprising p-xylene;
isomerizing the second xylene stream in a xylene isomerization reaction zone under xylene isomerization reaction conditions in the presence of a xylene isomerization catalyst to form a xylene isomerization effluent stream comprising a mixture of xylene;
recycling the xylene isomerization effluent stream to the xylene separation zone; and
recovering the p-xylene stream.

6. The process of claim 5 further comprising:
dealkylating the heavy alkylbenzene stream in a second dealkylation reaction zone comprising a second dealkylation reactor under second dealkylation conditions in the presence of a second dealkylation catalyst to form a dealkylated heavy alkylbenzene effluent stream comprising xylene, and benzene or toluene or both; or transalkylating the heavy alkylbenzene stream in a second transalkylation reaction zone comprising a second transalkylation reactor under second transalkylation conditions in the presence of a second transalkylation catalyst to form a transalkylated heavy alkylbenzene effluent stream comprising xylene, and benzene or toluene or both; and
optionally recycling the dealkylated heavy alkylbenzene effluent stream, the transalkylated heavy alkylbenzene effluent stream, or both to the alkylbenzene separation zone.

7. The process of claim 1 wherein the bottom stream from the dealkylation separation zone further comprises toluene or xylenol or both.

8. The process of claim 1 wherein providing the alkylphenol stream comprises:
providing a coal-derived feed stream to a first separation zone; and
separating the coal-derived feed stream in the first separation zone into the alkylphenol stream and at least one of a first phenol stream comprising phenol, and a first cresol stream comprising cresol.

9. The process of claim 8 wherein separating the coal-derived feed stream comprises separating the coal-derived feed stream by one or more of extraction, absorption, extractive distillation, crystallization, or membrane separation.

10. The process of claim 8 wherein the coal-derived feed stream comprises a portion of a low temperature coal tar stream, a medium temperature coal tar stream, a high temperature coal tar stream, a cresylic acid stream, or a crude phenolic mixture.

11. The process of claim 1 wherein the first dealkylation reaction conditions comprise one or more of: a temperature in a range of 50° C. to 700° C.; a pressure in a range of 0 MPa (g) to 5 MPa (g); a WHSV in a range of 0.1 to 300 hr⁻¹.

12. The process of claim 1 wherein the first transalkylation reaction conditions comprise one or more of: a temperature in a range of 50° C. to 600° C.; a pressure in a range of 0 MPa (g) to 15 MPa (g); and a WHSV in a range of 0.1 to 300 hr⁻¹.

13. The process of claim 1 wherein dealkylating the alkylphenol stream and the reactant stream takes place in the presence of steam, nitrogen, or a combination thereof in the dealkylation reaction zone.

14. The process of claim 1 wherein transalkylating the bottom stream and the reactant stream takes place in the presence of hydrogen, nitrogen, steam, or a combination thereof in the first transalkylation reaction zone.

15. The process of claim 1 wherein the dealkylation catalyst comprises a heterogeneous acid catalyst, or wherein the first transalkylation catalyst comprises a heterogeneous acid catalyst, or both.

16. The process of claim 1 wherein at least one of the first dealkylation reaction zone and the first transalkylation reaction zone comprises a fixed bed reactor, a moving bed reactor, an ebullated bed reactor, a fluidized bed reactor, a continuous catalyst regeneration (CCR) reactor, a semi-regenerative reactor, a batch reactor, a continuous stirred tank (CSTR) reactor, a slurry reactor, or combinations thereof.

17. A process for producing phenol from a coal-derived feed comprising:
providing an alkylphenol stream comprising alkylphenols having an alkyl chain having two or more carbon atoms;
dealkylating the alkylphenol stream in a first dealkylation reaction zone comprising a first dealkylation zone under first dealkylation reaction conditions in the presence of a first dealkylation catalyst to produce a first dealkylation effluent stream comprising light hydrocarbons, alkylbenzene, phenol, cresol, and unconverted alkylphenol;
separating the first dealkylation effluent stream in a dealkylation separation zone into at least a phenol stream comprising the phenol, a bottom stream comprising the unconverted alkylphenol, and at least one of a cresol stream comprising the cresol, a light hydrocarbon stream comprising the light hydrocarbons, and an alkylbenzene stream comprising the alkylbenzene;
transalkylating the bottom stream and a reactant stream comprising toluene or benzene or both in a first transalkylation reaction zone comprising a first transalkylation reactor under first transalkylation reaction conditions in the presence of a first transalkylation catalyst to produce a first transalkylation effluent stream comprising cresol, phenol, and alkylbenzene;
introducing the first transalkylation effluent stream into the dealkylation separation zone; and
recovering the phenol stream;
wherein the coal-derived feed stream comprises a portion of a low temperature coal tar stream, a medium temperature coal tar stream, a high temperature coal tar stream, a cresylic acid stream, or a crude phenolic mixture.

18. The process of claim 17 further comprising:
separating the cresol stream from the dealkylation separation zone in a cresol separation zone into a mixed cresol stream comprising p-cresol and o-cresol, and a m-cresol stream comprising m-cresol;
isomerizing the mixed cresol stream in a cresol isomerization reaction zone under cresol isomerization reaction conditions in the presence of a cresol isomerization catalyst to form a cresol isomerization effluent stream comprising a mixture of cresol;
recycling the cresol isomerization effluent stream to the cresol separation zone;
recovering the m-cresol stream comprising m-cresol;
separating the alkylbenzene stream from the dealkylation separation zone in an alkylbenzene separation zone into at least a recycle stream comprising one or more of benzene and toluene, a heavy alkylbenzene stream comprising alkylbenzene having an alkyl chain having 2 or more carbon atoms, and a mixed xylene stream comprising a mixture of xylenes and ethylbenzene;
recycling the recycle stream from the alkylbenzene separation zone to the first transalkylation reaction zone;
separating the mixed xylene stream in a xylene separation zone into a second xylene stream comprising ethylbenzene, o-xylene, and m-xylene, and a p-xylene stream comprising p-xylene;
isomerizing the second xylene stream in a xylene isomerization reaction zone under xylene isomerization reaction conditions in the presence of a xylene isomerization catalyst to form a xylene isomerization effluent stream comprising a mixture of xylene;
recycling the xylene isomerization effluent stream to the xylene separation zone;
recovering the p-xylene stream;
dealkylating the heavy alkylbenzene stream in a second dealkylation reaction zone comprising a second dealkylation reactor under second dealkylation conditions in the presence of a second dealkylation catalyst, to form a dealkylated heavy alkylbenzene effluent stream comprising xylene and benzene or toluene or both; or transalkylating the heavy alkylbenzene stream in a second transalkylation reaction zone comprising a second transalkylation reactor under second transalkylation conditions in the presence of a second transalkylation catalyst to form a transalkylated heavy alkylbenzene effluent stream comprising xylene and benzene or toluene or both; and
recycling the dealkylated heavy alkylbenzene effluent stream, the transalkylated heavy alkylbenzene effluent stream, or both to the alkylbenzene separation zone.

19. The process of claim 17 wherein providing the alkylphenol stream comprises:
providing a coal-derived feed stream to a first separation zone; and
separating the coal-derived feed stream in the first separation zone into the alkylphenol stream and at least one of a first phenol stream comprising phenol, and a first cresol stream comprising cresol.

20. The process of claim 17:
wherein the first dealkylation reaction conditions comprise one or more of: a temperature in a range of 50° C. to 700° C.; a pressure in a range of 0 MPa (g) to 5 MPa (g); a WHSV in a range of 0.1 to 300 hr⁻¹; or
wherein the first transalkylation reaction conditions comprise one or more of: a temperature in a range of 50° C. to 600° C.; a pressure in a range of 0 MPa (g) to 15 MPa (g); and a WHSV in a range of 0.1 to 300 hr⁻¹; or both.
